(19) United States
(12) Patent Application Publication
(10) Pub. No.: US 2009/0099141 A1
(43) Pub. Date: Apr. 16, 2009

(54) COMPOSITIONS AND METHODS FOR TREATING VITAMIN D DEFICIENCIES

(76) Inventor: James Grote, Pittsfield, IL (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 12/341,277

(22) Filed: Dec. 22, 2008

Related U.S. Application Data
(63) Continuation-in-part of application No. 11/063,037, filed on Feb. 20, 2005.

(51) Int. Cl. A61K 31/593; C07C 401/00; A61P 3/02

(52) U.S. Cl. 514/167; 552/653

(57) ABSTRACT

The present invention provides for: (a) an oral dosage of vitamin D3 for humans sufficient to maintain a serum level above a minimally sufficient level and below a toxic level when administered according to a schedule of at least one dosage a week up to at least one dosage a day, (b) methods for administering the oral dosages and (c) a kit involving a blister pack and a plurality of oral dosages with instructions for administering the dosages and a location on the blister pack for recording information related to the administration of each dosage, the name of the person receiving the dosages and additional relevant information.

### REFERENCE TO RELATED APPLICATIONS

This application is a continuation-in-part of U.S. patent application Ser. No. 11/063,037, the entire contents of which are hereby incorporated herein by reference.

### FIELD OF THE INVENTION

The present invention relates generally to dosage forms of vitamin D3, and more particularly to dosage forms of vitamin D3 that are effective for treating adult humans without presenting a risk of vitamin D3 toxicity when the recommended administration regime is not followed.

### BACKGROUND OF THE INVENTION

Vitamin D3 is an essential hormone that is formed in the skin after exposure to UV radiation in sunlight. It may also be provided from a fortified diet or from dietary supplements, but with few exceptions there is little or no vitamin D3 in the foods humans normally eat.

In the body, vitamin D is hydroxylated in the liver to form 25(OH)D which is further hydroxylated in kidneys to give 25(OH)₂D, the active form of the vitamin. Serum levels of 25(OH)₂D are difficult to measure though, and increased levels of parathyroid hormone can elevate the level of 25(OH)₂D, masking a deficiency. Because serum levels of the precursor 25(OH)D are readily measured, it is generally considered to be the better indicator of vitamin D levels in humans.

Historically, vitamin D provided in cod liver oil was first utilized to treat a deficiency disease called rickets, a condition that affects the development of a child's skeletal system. More recently, it has become understood that vitamin D plays a role in a myriad of human disease states involving at least the kidney, pancreas, intestine, liver, thyroid, parathyroid, bones, colon, prostate, lungs, and skin. In addition, vitamin D is known to play a significant role in the reduction of certain cancers, multiple sclerosis, and hypertension.

The recommended daily allowance of vitamin D3 was initially set at 10 μg (400 IU) because that amount approximated the amount of the vitamin in a teaspoon of cod liver oil. That dosage level has proven effective for most indications when taken daily, and formulations of vitamin D are currently available in that dosage. Current recommended doses have not proven effective, however, for protecting against osteoporosis or over-stimulation of the parathyroid gland.

In spite of the known benefits of taking daily doses of vitamin D, many patients either forget or find it inconvenient to take a daily dose. For such patients, the therapeutic benefits of vitamin D are not obtained since an appropriate level of 25(OH)D is not generally maintained in the blood stream when 10 μg (400 IU) formulations of vitamin D are taken less frequently than daily.

At high dose levels however, vitamin D is known to cause hypercalcemia as excessive calcium is taken into the bloodstream from the intestine and bone. This results in deposition of calcium and phosphorus in soft tissues all over the body, with particular damage to the heart, blood vessels and kidneys. Since a dosage level designed for weekly or monthly administration may be taken daily by some patients, a risk of vitamin D toxicity would arises if the weekly or monthly dosage level were not designed to account for varying frequencies of administration.

A need therefore exists for alternative dosage formulations of vitamin D that address the problems associated with varying intervals of administration. The present invention satisfies that need.

### SUMMARY OF THE INVENTION

One form of the present invention contemplates a composition comprising an oral vitamin dosage form for maintaining an adult human's serum level of 25-hydroxycholecalciferol [25(OH)D] at a level of between about 40 nmole/L and about 200 nmole/L provided said oral dosage is administered according to a schedule of at least about once a week and not more frequently than about once a day. The oral dosage comprises about 250 μg of vitamin D3. The dosage can be formulated as a solution or suspension in an acceptable solvent such as ethanol or as a solid. The preferred oral dosage is a single tablet, capsule or gelcap containing about 250 μg of vitamin D3.

A further aspect of this invention is a method of maintaining a serum level of 25(OH)D in a human adult above a minimally deficient level within a range of between about 40 nmole/L and about 200 nmole/L. The method comprises:
(a) obtaining a dosage of about 250 μg of vitamin D3;
(b) introducing said dosage into the intestinal lumen of said adult by having said adult swallow said dosage;
(c) repeating said introducing according to a schedule of at least about once a week up to about once a day.
   Depending on the repeating schedule, a generally constant serum level of 25(OH)D is obtained within about 30 to 90 days.

Another aspect of the present invention is a method of increasing a human adult's serum level of 25(OH)D, the method comprising:
(a) obtaining a tablet, capsule, or gelcap containing about 250 μg of vitamin D3 substantially free of calcium;
(b) introducing said tablet, capsule, or gelcap into the intestinal lumen of said adult by having said adult swallow said tablet, capsule, or gelcap;
(c) repeating said introducing according to a schedule of at least about once a week up to about once a day; and
(d) achieving an increase in the serum level of 25(OH)D of at least about 15 nmole/L.

A still further aspect of the present invention is a kit comprising a blister pack containing the a plurality of oral dosages of vitamin D3 sufficient to maintain an adult human's serum level of 25(OH)D at a level between about 40 to about 200 nmol/L when administered according to a prescribed schedule of from about one dosage a week to about one dosage a day, wherein said dosage comprises about 250 μg of vitamin D3. The blister pack is formatted to assist in administering said dosages according to said prescribed schedule and protects the oral dosages from degradation upon contact with moisture and air. Preferred blister packs additionally provide a surface region to record administration details such as name, date and time administered, person administering the dosage and the like.

Related objects and advantages of the present invention will be apparent from the following description.

### BRIEF DESCRIPTION OF THE DRAWINGS

FIG. 1 illustrates a blister pack containing four (4) ampoules containing dosages of 250 μg of vitamin D3 in absolute ethanol with administration instructions providing for the contents of one (1) ampoule to be taken each week.

FIG. 2 illustrates a blister pack containing 31 tablets, each containing 250 μg of vitamin D3 with administration instructions providing for one tablet to be taken each day.

FIG. 3 illustrates a blister pack containing 15 gelcaps, each containing 250 μg of vitamin D3 with administration instructions providing for one gelcap to be taken every two days and additionally having a surface region to record administration details.

### DESCRIPTION OF THE PREFERRED EMBODIMENTS

For the purposes of promoting an understanding of the principles of the invention, reference will now be made to certain preferred embodiments and specific language will be used to describe the same. It will nevertheless be understood that no limitation of the scope of the invention is thereby intended, such alterations and further modifications of the preferred embodiments being contemplated as would normally occur to one skilled in the art to which the invention relates.

During the summer months, healthy adults spending some time outside in direct sunlight generally have sufficient levels of vitamin D as evidenced by their serum concentration of 25(OH)D formed from vitamin D3 in the liver. However, age, disease and minimal exposure to sunlight can reduce the concentration of 25(OH)D sufficiently to cause increased levels of parathyroid hormone, osteoporosis and other physical problems. Insufficient exposure to sunlight can result from an individual's occupation, geographical location, physical condition and age and cause serum levels of 25(OH)D to fall below a minimally sufficient level of about 38 nmole/L to as low as about 10 nmole/L.

The current recommended dietary allowance of vitamin D for infants and children is 10 μg or 400 IU, which approximates the amount of vitamin D in a teaspoon of cod-liver oil used in the treatment of rickets. For adults, the recommended dietary allowance is only about 5 μg or 200 IU.

Although there is no general consensus of the optimal level of vitamin D3 intake, current evidence suggests that intake should be sufficient to cause a serum concentration of 25(OH)D of at least about 38 nmole/L to about 100 nmole/L in order to minimize the serum level of parathyroid hormone and to prevent osteoporosis fracture in aging adults.

Although there is some dispute about what the toxic level of vitamin D3 is, no credible reports of toxic effects exist for dosages of vitamin D3 sufficient to raise serum levels of 25(OH)D to a level of up to about 200 to 250 nmole/L. Current over the counter formulations typically contain about 10 μg (400 I.U.) of vitamin D3 and are insufficient to significantly raise the serum level of 25(OH)D in an adult.

One aspect of the present invention provides for an oral dosage of 200 μg to 300 μg, preferably 225 μg to 2750 μg, and most preferably about 250 μg or 10,000 IU of vitamin D3 capable of increasing the serum level of 25(OH)D in an adult human by at least about 25 nmole/L to a level of at least about 40 nmole/L when administered according to a schedule of about one dosage a week and to a level of about 200 nmole/L when administered according to a schedule of about one dosage a day. For preparing the oral dosage of the present invention, pharmaceutically acceptable carriers can be either solid or liquid.

Solid form preparations include powders, tablets, pills, capsules, cachets, dispersible granules and the like. A solid carrier can be one or more substances which may also act as diluents, flavoring agents, binders, preservatives, tablet disintegrating agents or an encapsulating material. Formulations with solid carriers will generally involve tablets, capsules and the like. Liquid carriers must be capable of dissolving or dispersing the vitamin to form a solution, suspension or the like without causing degradation of the vitamin. Liquid formulations involving solutions and dispersions will generally be contained in bottles, vials, ampules, gelcaps and the like.

For a tablet or capsule form, vitamin D3 can be combined with an oral, non-toxic, pharmaceutically acceptable, inert carrier such as lactose, starch, sucrose, glucose, methyl cellulose, magnesium stearate, mannitol, sorbitol and the like. Although minor amounts of inert carriers such as dicalcium phosphate and calcium sulfate can be employed, preferred embodiments are substantially free of calcium.

For a liquid form contained in a bulk container, ampules, or a gelcap, vitamin D3 can be dissolved or suspended in non-toxic, pharmaceutically acceptable, inert carrier such as dry ethanol, dry glycerol and the like. Both solid and liquid formulations should be protected from exposure to light and moisture to minimize degradation of the vitamin. The more preferred embodiments of the oral dosage are tablets, capsules and gelcaps.

When properly administered, the invention can raise and maintain a satisfactory and beneficial serum level of 25(OH)D in a human adult without a risk of causing toxic serum levels of vitamin D. Toxic levels are difficult to reach even if the dosage form is taken in excess. The invention additionally relates to methods of administering the dosage form of the vitamin to raise and maintain the desired and beneficial serum levels of 25(OH)D. In a preferred aspect, a kit is provided that contains the dosage form of the vitamin in a blister pack formatted with instructions to facilitate the vitamin's administration.

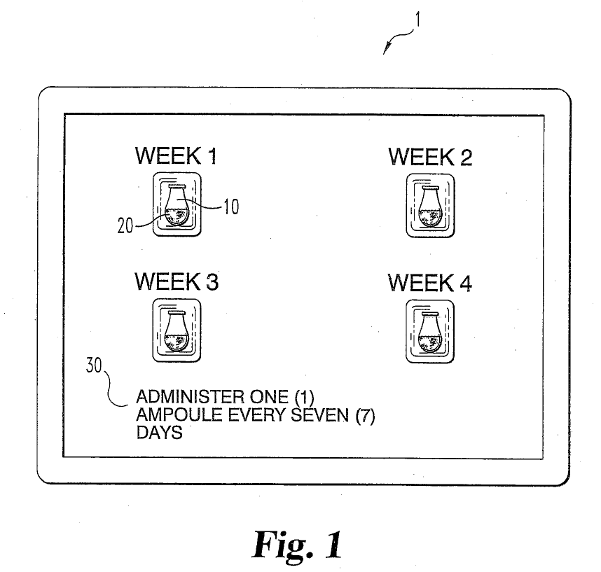

With reference to FIG. 1, a kit is illustrated comprising a blister pack 1 designed to contain four (4) ampules 10 containing a liquid formulation 20 containing 250 μg (10,000 IU) of vitamin D3 in absolute ethanol. The blister pack 1 is formatted with instructions 30 to assist in administering a single dosage once a week.

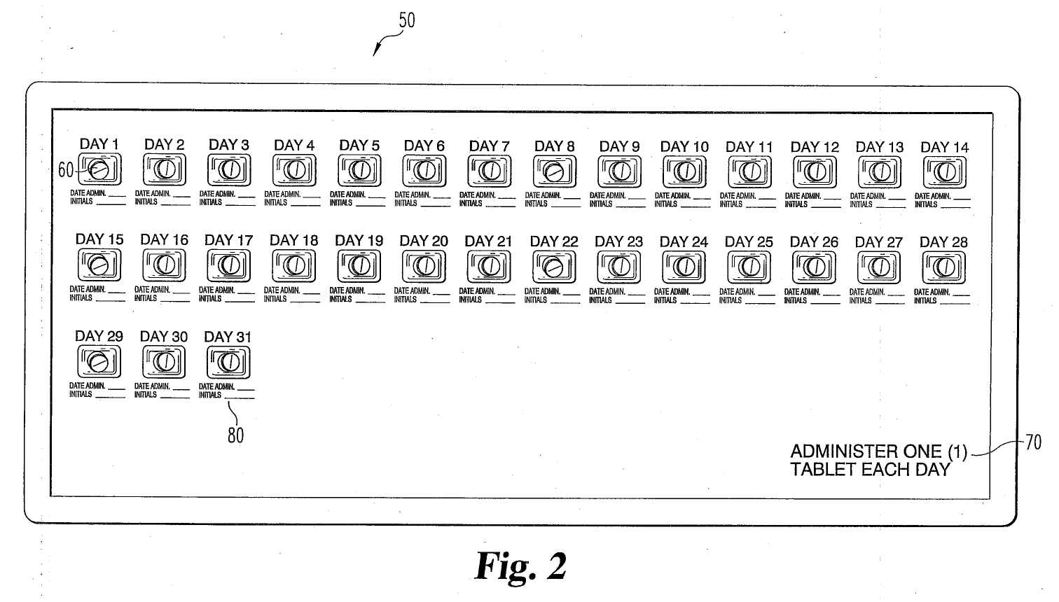

With reference to FIG. 2, a kit is illustrated comprising a blister pack 50 containing 30 tablets 60, each tablet containing 250 μg (10,000 IU) of vitamin D3. The blister pack 50 is formatted with instructions 70 to assist in administering the dosages once each day and has regions 80 for designating administrative details, such as the name of the person receiving the vitamin, when a dosage was taken and who administered the dosage. Blister packs according to the present embodiment of the invention will contain from about 28 to about 31 tablets to account for the variation in the number of days in the different months of the year.

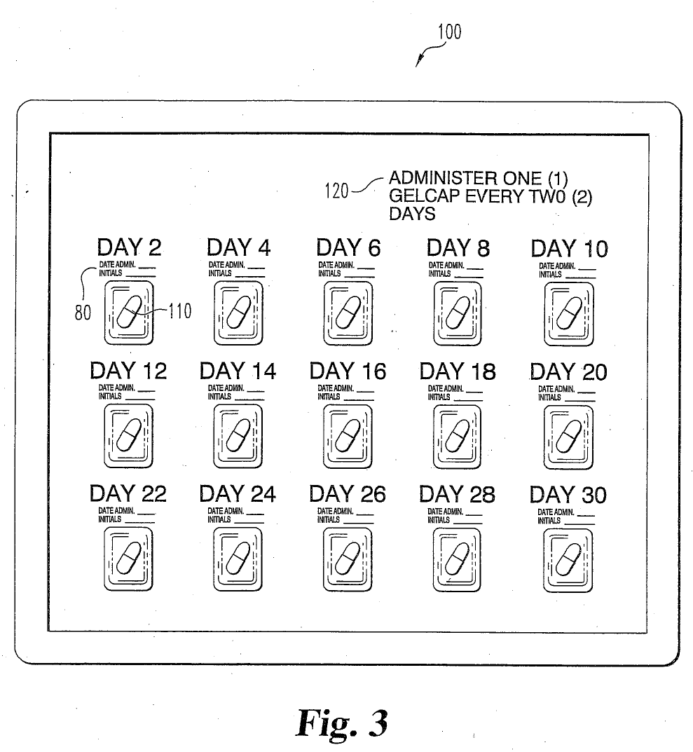

With reference to FIG. 3, a kit is illustrated comprising a blister pack 100 containing 15 gelcaps 110, each gelcap containing 250 μg (10,000 IU) of vitamin D3. The blister pack 100 is formatted with instructions 120 to administer a gelcap once every two days and has regions 80 for designating when the gelcap was administered and who administered the gelcap. Kits of the type illustrated by FIG. 3 can help ensure that the vitamin is taken according to a prescribed schedule and provide documentation of how the vitamin was administered. This is particularly important for elderly adults taking several medications according to different schedules or receiving their vitamin along with other medications from a healthcare worker having the responsibility of providing medications to a large number of persons.

In further embodiments the vitamin D3 dosage form may be provided with an enteric coating. For example, capsules of 250 μg (10,000 IU) vitamin D3 may be provided with an enteric coating comprising one or more members of the group consisting of cellulose acetate phthalate (CAP), methyl acrylate-methacrylic acid copolymers, cellulose acetate succinate, hydroxy propyl methyl cellulose phthalate, polyvinyl acetate phthalate (PVAP), and methyl methacrylate-methacrylic acid copolymers.

Variations of these embodiments can similarly be utilized to contain tablets, ampules, capsules, gelcaps or the like where one or more tablets, ampules, capsules, gelcaps or the like are administered once a week, once each day or at some intermediate time interval. Blister packs can contain dosages for a single month as illustrated or can contain dosages for up to about a year and can have the form of a single card, a fold-out arrangement or notebook arrangement having several pages of blister packs. Exemplary blister packs are known to the art, as shown, for example, by U.S. Pat. Nos. 4,192,422 and 4,817,819, both of which are incorporated herein by reference.

The following examples illustrate the use of applicant's invention to elevate the level of 25(OH)D in an adult by about 25 nmole/L and to maintain the level of 25(OH)D at a desired level between about 38-40 nmole/L up to at least about 200 nmole/L.

### EXAMPLE 1

During the winter months an adult male living in a northern region of the United States receiving no vitamin D supplements other than through his normal diet is found to have a serum level of 25(OH)D of about 25 nmole/L. The male is provided with four (4) blister packs, each pack containing four (4) 250 μg (10,000 IU) of vitamin D3. The blister pack has instructions to take one tablet by mouth once a week. The male is instructed to take all of the tablets according to instructions provided on the pack. Blood samples are drawn each week just before a tablet is taken and the serum levels of 25(OH)D are determined. Serum levels of 25(OH)D can be determined by the Nichols method (catalog no. 40-2135; Nichols Institute Diagnostics). Within approximately two months, serum levels increase by about 15 to about 20 nmole/L to a level of at least about 40 nmole/L.

### EXAMPLE 2

During the winter months an adult female living in a northern region of the United States receiving no vitamin D supplements other than through her normal diet is found to have a serum level of 25(OH)D of about 18 nmole/L. The female is provided with four (4) blister packs, each pack having one tablet for each day of the month [about thirty (30) tablets], and each tablet containing 250 μg (10,000 IU) of vitamin D3. The blister pack has instructions to take one tablet by mouth once a day, noting on the pack, where indicated, the date and time each tablet is taken. The female is instructed to take all of the tablets according to instructions provided on the pack. Blood samples are drawn after one (1) week and each week thereafter at approximately the same time and the serum levels of 25(OH)D are determined. Serum levels of 25(OH)D can be determined by the Nichols method (catalog no. 40-2135; Nichols Institute Diagnostics). Within approximately two months, serum levels increase by at least 100 nmole/L to a level of at least about 120 nmole/L and, within about 4 months, increase by at least about 180 nmole/L to a level of at least about 200 nmole/L.

### EXAMPLE 3

During the winter months an adult male living in a northern region of the United States receiving no vitamin D supplements other than through his normal diet is found to have a serum level of 25(OH)D of about 35 nmole/L. The female is provided with four (4) blister packs, each pack containing fifteen (15) 250 μg (10,000 IU) of vitamin D3. The blister pack has instructions to take one tablet by mouth once every other day, noting on the pack, where indicated, the date and time each tablet is taken. The male is instructed to take all of the tablets according to instructions provided on the pack. Blood samples are drawn each week at approximately the same time and the serum levels of 25(OH)D are determined. Serum levels of 25(OH)D can be determined by the Nichols method (catalog no. 40-2135; Nichols Institute Diagnostics). Within approximately one-month serum levels increase by at least about 60-70 nmole/L to a level of at least about 100 nmole/L and within about 4 months increase by at least about to a level of about 120 nmole/L.

While certain aspects of the invention have been described in detail in the drawings and foregoing description, the same are to be considered illustrative of the claimed invention and not limiting, it being understood that all variations and modifications that come within the spirit of the invention are desired to be protected.

What is claimed:

1. A method of obtaining and maintaining a serum level of 25(OH)D in a human adult within a range of between about 40 nmole/L and about 200 nmole/L, the method comprising:
(a) obtaining a dosage of about 250 μg of vitamin D3, wherein said dosage is provided with an enteric coating;
(b) introducing said dosage into the intestinal lumen of said adult by having said adult swallow said dosage;
(c) repeating said introducing according to a schedule of at least about once a week up to about once a day.

2. The method of claim 1, wherein said dosage is contained within a tablet, a capsule, or a gelcap.

3. The method of claim 2, wherein said dosage is substantially calcium free.

4. The method of claim 3, wherein said repeating is about once a week.

5. The method of claim 3, wherein said repeating is about once every two days.

6. The method of claim 3, wherein said repeating is about once a day.

7. A method of increasing a human adult's serum level of 25(OH)D, the method comprising:
(a) obtaining a tablet, capsule, or gelcap containing about 250 μg of vitamin D3 substantially free of calcium;
(b) introducing said tablet, capsule, or gelcap into the intestinal lumen of said adult by having said adult swallow said tablet, capsule, or gelcap;
(c) repeating said introducing according to a schedule of at least about once a week up to about once a day; and
(d) achieving an increase in the serum level of 25(OH)D of at least about 15 nmole/L.

8. The method of claim 7, wherein said repeating is at least about once a week.

9. The method of claim 8, wherein said repeating is at least about once a day.

10. A kit comprising:
(a) a plurality of oral dosages of vitamin D3 sufficient to maintain an adult human's serum level of 25(OH)D at a level of from about 40 to about 200 nmol/L when administered according to a prescribed schedule of from about one dosage a week to about one dosage a day, wherein said dosage comprises about 250 μg of vitamin D3, and
(b) a blister pack containing said plurality of dosages, said blister pack formatted to assist in administering said dosages according to said prescribed schedule.

11. The kit of claim 10, wherein said plurality of dosages comprises a month's supply.

12. The kit of claim 11, wherein said prescribed schedule requires administering about one dosage a week.

13. The kit of claim 12, wherein said prescribed schedule requires administering about one dosage a day.

14. The kit of claim 10, wherein said dosages are substantially calcium free.

15. The kit of claim 10, wherein said dosage is contained within a tablet, a capsule, or a gelcap.

16. The kit of claim 10, wherein said blister pack has a designated surface region to record administration details.

17. A composition which comprises an oral vitamin dosage form for maintaining an adult human's serum level of 25(OH)D at a level of between about 40 nmole/L and about 200 nmole/L provided said oral dosage is administered according to a schedule of at least about once a week and not more frequently than about once a day, wherein said oral dosage comprises about 250 μg of vitamin D3.

18. The composition of claim 15, wherein said composition is substantially free of calcium.

19. The composition of claim 16, wherein said dosage is contained within a tablet, a capsule, or a gelcap.

20. A method of obtaining and maintaining a serum level of 25(OH)D in a human adult within a range of between about 40 nmole/L and about 200 nmole/L, the method comprising:
(a) obtaining a dosage of about 250 μg of vitamin D3;
(b) introducing said dosage into the intestinal lumen of said adult by having said adult swallow said dosage;
(c) repeating said introducing according to a schedule of at least about once a week up to about once a day.
